(19) United States
(12) Patent Application Publication
(10) Pub. No.: US 2003/0225029 A1
(43) Pub. Date: Dec. 4, 2003

(54) Dosing regimen for gemcitabine HCV therapy

(76) Inventor: Lieven J. Stuyver, Snellville, GA (US)

Correspondence Address: KING & SPALDING, 191 PEACHTREE STREET, N.E., ATLANTA, GA, 30303-1763, US

(21) Appl. No.: 10/367,388

(22) Filed: Feb. 14, 2003

Related U.S. Application Data
(60) Provisional application No. 60/357,411, filed on Feb. 14, 2002.
(60) Provisional application No. 60/358,140, filed on Feb. 20, 2002.

(51) Int. Cl. A61K 31/7072; A61K 31/513

(52) U.S. Cl. 514/49; 514/269

(57) ABSTRACT

A dosage regiment for the treatment of a Flaviviridae infection, including a hepatitis C viral infection, that includes administering gemcitabine (or its salt, prodrug or derivative, as described herein) in a dosage range of approximately 50 mg/m² to about 1300 mg/m² per day for between one and seven days (e.g. 1, 2, 3, 4, 5, 6, or 7 days) followed by cessation of therapy. Viral load is optionally monitored over time, and after cessation, viral rebound is monitored. Therapy is not resumed unless a significant viral load is again observed, and then therapy for 1-7 days and more preferred, 1, 2 or 3 days, is repeated. This therapy can be continued indefinitely to monitor and maintain the health of the patient.

This application claims priority to U.S. patent application No. 60/357,411, filed on Feb. 14, 2002, and U.S. patent application No. 60/358,140, filed on Feb. 20, 2002.

### FIELD OF THE INVENTION

The present invention is a method and dosing regimen for the treatment of a flavivirus or pestivirus, notably hepatitis C virus (HCV), using gemcitabine or its pharmaceutically acceptable salt or prodrug or a derivative thereof.

### BACKGROUND OF THE INVENTION

Gemzar® (gemcitabine HCl) is a pyrimidine antimetabolite with antitumor activity against leukemias and a variety of solid tumors (e.g., pancreatic, non-small cell lung cancer, ovarian, breast, mesothelioma, etc.). Gemcitabine is a nucleoside analogue of the formula β-D-2′,2′-difluorocytidine (see structure below). Gemcitabine was originally investigated for its antiviral effects but has since been developed as an antineoplastic agent. (Delong, D. C., L. W. Hertel, and J. Tang. Antiviral activity of 2′,2′ Difluorodeoxycytidine; American Society of Microbiology. 1986.) Gemcitabine has been approved by the Food and Drug Administration (FDA) for the following indications: (1) in combination with cisplatin as first-line treatment for patients with inoperable, locally advanced (Stage IIIA or IIIB) or metastatic (Stage IV) non-small cell lung cancer (NSCLC), (2) as a first-line treatment for patients with locally advanced (nonresectable Stage II or Stage III) or metastatic Stage (IV) adenocarcinoma of the pancreas and (3) as a second-line therapy for pancreatic cancer in patients previously treated with 5-fluorouracil (5-FU).

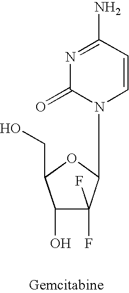

Gemcitabine (dFdC) is a cell cycle specific agent that primarily targets cells undergoing DNA synthesis (S-phase). Gemcitabine is metabolized intracellularly by the rate limiting enzyme deoxycytidine kinase (dCK) to its monophosphate form (dFdCMP). (Heinemann, V., et al., Comparison of the Cellular Pharmacokinetics and Toxicity of 2′,2′-Difluorodeoxycytidine and 1-Beta-D-Arabinofuranosylcytosine. Cancer Res. 1988. 48(14): 4024-31). Subsequent phosphorylation by other nucleoside kinases leads to the formation of the active metabolites dFdCDP and dFdCTP. The cytotoxicity of gemcitabine is attributed to a combination of actions by the diphosphate and triphosphate metabolites that enhance the lethal effects of this agent. These actions are summarized in FIG. 1. First, dFdCDP inhibits ribonucleotide reductase (pathway 1) and this reduces the concentration of cellular deoxynucleotides (e.g., deoxycytidine triphosphate, dCTP) required for DNA replication (FIG. 1; Self-Potentiating Actions of Gemcitabine and DNA repair). Reduced cellular dCTP concentrations that result from the inhibition of ribonucleotide reductase favor dFdCTP analog incorporation into DNA, an event critical for gemcitabine-induced lethality (pathway 2). (Huang, P. and W. Plunkett, Fludarabine-and Gemcitabine-Induced Apoptosis: Incorporation Of Analogs Into DNA Is A Critical Event. Cancer Chemother Pharmacol, 1995. 36(3):181-8; Huang, P. and W. Plunkett, Induction Of Apoptosis By Gemcitabine. Semin Oncol, 1995. 22(4 Suppl 11):19-25.). Reduced cellular dCTP levels also increase the rate of gemcitabine phosphorylation because high dCTP levels inhibit the rate limiting enzyme dCK (pathway 3). In contrast to its inhibitory effect on dCK, dCTP is a cofactor required for the activity of dCMP deaminase, the rate-limiting enzyme for elimination of gemcitabine nucleotides from the cell (pathway 4). The cytotoxic metabolite dFdCTP directly inhibits dCMP deaminase (pathway 5). (Xu, Y. Z. and W. Plunkett, Modulation Of Deoxycytidylate Deaminase In Intact Human Leukemia Cells Action of 2′,2′-difluorodeoxycytidine. Biochem Pharmacol, 1992. 44(9): 1819-27). And finally, at high, intracellular concentrations FdCTP inhibits CTP synthetase (pathway 6) thereby blocking the synthesis of CTP, and consequently, that of dCTP as well. (Heinemann, V., et al., Gemcitabine: A Modulator Of Intracellular Nucleotide And Deoxynucleotide Metabolism. Semin Oncol, 1995. 22(4 Suppl 11):11-8).

Gemcitabine is a good substrate for phosphorylation by dCK to the monophosphate form dFdCMP, demonstrating a Km of 3.6 μmol/L with a substrate efficiency (Vmax/Km) similar to deoxycytidine (Km=1.6 μmol/L as determined using a partially purified enzyme from Chinese Hamster ovary (CHO) cells. (Heinemann, V., et al., Comparison Of The Cellular Pharmacokinetics And Toxicity Of 2′,2′-Difluorodeoxycytidine And 1-Beta-D-Arabinofuranosylcytosine. Cancer Res, 1988. 48(14): 4024-31). Phosphorylation of gemcitabine is essential for its biological activity and cells that lack dCK are not affected by gemcitabine. Studies with radioactive precursors of DNA, RNA and protein synthesis demonstrated that the effects of gemcitabine are primarily directed at DNA. (Plunkett, W., et al., Gemcitabine: Preclinical Pharmacology And Mechanisms Of Action. Semin Oncol, 1996. 23(5 Suppl 10):3-15). Model systems of DNA synthesis confirmed that the triphosphate, dFdCTP, is incorporated into growing DNA primer strands by human DNA polymerases a and ε. (Huang, P., et al., Action Of 2′,2′-Difluorodeoxycytidine On DNA Synthesis. Cancer Res, 1991. 51(22):6110-7) with each polymerase showing a 20-fold preference for the normal nucleotide (dCTP). Uniquely, incorporation of gemcitabine is followed by the addition of one more nucleotide before DNA polymerase is inhibited. When placed at the penultimate position, excision of dFdCMP by the 3→5′ proofreading exonuclease proceeds at a much slower rate than excision of dCMP. This phenomenon described as “masked chain termination” improves the ability of gemcitabine to inhibit DNA replication and repair and provides a mechanism for synergism of gemcitabine with DNA damaging agents (e.g., cisplatin).

Gemcitabine is a good substrate for intracellular cytidine deaminase (Km=96 μM), which is the enzyme responsible for the rapid metabolic clearance of gemcitabine via biotransformation to the deamination product 2′,2′-difluorodeoxyuridine (dFdU) during clinical use. (Bouffard, D. Y., J. Laliberte, and R. L. Momparler, Kinetic Studies On 2′,2′-Difluorodeoxycytidine (Gemcitabine) With Purified Human Deoxycytidine Kinase And Cytidine Deaminase. Biochem Pharmacol, 1993. 45(9):1857-61). Gemcitabine is rapidly deaminated in the blood, liver, kidneys, and other tissues. Gemcitabine disposition was evaluated in 5 human subjects who received a single dose of radiolabeled drug 1000 mg/m² by 30 min infusion. Gemcitabine (<10%) and the inactive metabolite dFdU accounted for 99% of the excreted dose. The metabolite dFdU was also detected in the plasma and gemcitabine plasma protein binding was negligible.

The pharmacokinetics of gemcitabine was examined in 353 patients (2/3 men) with various solid tumors. Pharmacokinetic parameters were determined using data from patients treated for varying durations of therapy administered at weekly intervals with periodic rest weeks and using both short (<70 min) and long infusions (70 to 285 min). The total gemcitabine dose administered ranged from 500 to 3600 mg/M². Gemcitabine pharmacokinetics are linear and described by the 2-compartment model. Elimination is dependent on renal excretion and clearance was influenced by age and gender. Population pharmacokinetic analyses of combined single and multiple dose studies determined that the volume of distribution of gemcitabine was significantly influenced by duration of infusion and gender. Gemcitabine half-life after short infusion ranged from 32-92 min and the value for long infusions varied from 245 to 638 min. These data reflected a greater volume of distribution with longer infusions. Volume of distribution was 50 L/m² following short infusions (<70 min), indicating that gemcitabine is not extensively distributed in the tissues. Conversely, volume of distribution increased to 370 L/m² after long infusions, reflecting a slow equilibration of gemcitabine within the tissue compartment. The metabolite did not accumulate with weekly dosing but its elimination depends on renal excretion and dFdU levels may be influenced by renal impairment.

The effects of significant renal or hepatic insufficiency on gemcitabine disposition have not been assessed. The active metabolite dFdCTP can be extracted from peripheral blood mononuclear cells and the terminal phase half-life of dFdCTP from mononuclear cells ranges from 1.7 to 19.4 hours.

Importantly, the maximum tolerated dose (MTD) is heavily dependent on schedule and frequency of infusion. (Boven, E., et al., The Influence Of The Schedule And The Dose Of Gemcitabine On The Antitumour Efficacy In Experimental Human Cancer. Br J Cancer, 1993. 68(1):52-6). Prolongation of infusion time beyond 60 min and more frequent than weekly dosing has been shown to increase gemcitabine-related toxicity. Typically, myelosuppression is the dose-limiting toxicity manifested by leukopenia, thrombocytopenia, and anemia. Patients should be monitored for myelosuppression during therapy because dosage adjustments for hematologic toxicity are frequently needed. Other toxicities associated with gemcitabine include stomatitis, nausea and vomiting, fever, rash, mild parasthesias, mild alopecia, flu-like symptoms (i.e., fever, chills, myalgia, cough, and headache) dypsnea, edema, mild proteinuria and hematuria, transient elevation of one or both serum transaminases, and diarrhea. Two clinical trials evaluated the efficacy of gemcitabine in patients with locally advanced or metastatic pancreatic cancer. The first trial compared gemcitabine with 5-FU in patients who had received no prior chemotherapy and the second trial evaluated patients who had received prior therapy with 5-FU or a 5-FU-containing regimen. In both studies gemcitabine was administered at a dose of 1000 mg/m² by 30 min infusion once weekly for 7 consecutive weeks (or until toxicity required withholding a dose) followed by one week of rest from treatment. Subsequent cycles consisted of weekly infusions for three consecutive weeks followed by one week of rest. The primary efficacy parameter in these studies was based on clinical benefit response defined and measured by improvements based on analgesic consumption, pain intensity, performance status and weight change. The first study was a multicenter, prospective, single blind, randomized comparison of gemcitabine and 5-FU in patients with locally advanced or metastatic pancreatic cancer. (Burris, H. A., 3rd, et al., Improvements In Survival And Clinical Benefit With Gemcitabine As First-Line Therapy For Patients With Advanced Pancreas Cancer: A Randomized Trial. J. Clin Oncol, 1997. 15(6):2403-13). Gemcitabine was associated with statistically significant increases in clinical benefit response, survival, and time to disease progression compared to 5-FU with 63 patients evaluated in each treatment arm. The second trial was a multicenter open label study of gemcitabine in 63 patients previously treated with 5-FU or a 5-FU-containing regimen. (Rothenberg, M. L., et al., A Phase II Trial Of Gemcitabine In Patients With 5-FU-Refractory Pancreas Cancer. Ann Oncol, 1996. 7(4):347-53). The study showed a clinical benefit response rate of 27% with a median survival of 3.9 months.

Data from two randomized studies (657 patients) support the use of gemcitabine in combination with cisplatin for the first-line treatment of patients with locally advanced or metastatic NSCLC. One study compared gemcitabine plus cisplatin versus cisplatin alone and the second study evaluated gemcitabine plus cisplatin versus etoposide plus cisplatin. A total of 522 subjects were evaluated in the first study. (Mitchell, P. L., Quality Of Life And Cisplatin-Gemcitabine Chemotherapy. J. Clin Oncol, 2000. 18(14): 2791-2). Gemcitabine (1000 mg/m²) was administered on days 1, 8 and 15 of a 28-day cycle of cisplatin 100 mg/m² administered on day 1 of each cycle. Median survival time and median time to disease progression were significantly greater in the gemcitabine plus cisplatin treatment arm compared to cisplatin alone. The objective response rate was 26% in the gemcitabine plus cisplatin treatment arm compared to 10% for cisplatin. In the second multicenter study 135 patients with stage IIIB or Stage 1V NSCLC patients were treated with gemcitabine (1250 mg/m²) on days 1 and 8 and cisplatin 100 mg/m2 on day 1 of a 21-day cycle or with etoposide 100 mg/m² I.V. on days 1, 2, and 3 and cisplatin 100 mg/m² on day 1 of a 21-day cycle. (Cardenal, F., et al., Randomized Phase III Study Of Gemcitabine-Cisplatin Versus Etoposide-Cisplatin In The Treatment Of Locally Advanced Or Metastatic Non-Small-Cell Lung Cancer. J. Clin Oncol, 1999. 17(1):12-8). There was no significant difference in survival between the two treatment arms. Nevertheless, median time to disease progression and objective response rates were significantly greater in the gemcitabine plus cisplatin treatment arm compared to etoposide plus cisplatin.

Several cases of acute respiratory distress syndrome (ARDS) related to Gemcitabine treatment have been reported since 1997. These cases are associated with significant morbidity and mortality (Sabria-Trias et al. Rev Mal Respir. 2002. 19:645-7; Gupta et al. Am J Clin Oncol. 2002; 25(1):96-100). Gemcitabirie pulmonary toxicity has been linked to the detection of Gemcitabine-induced systemic capillary leak syndrome (SCLS), a rare disorder with a high mortality rate, characterized by rapidly developing edema, weight gain and hypotension, hemoconcentration and hypoproteinemia, is caused by sudden, reversible capillary hyperpermeability with a rapid extravasation of plasma from the intravascular to the interstitial space. Recent evidence suggests that gemcitabine SCLS is the pathogenic mechanism for the pulmonary toxicity of gemcitabine (De Pas et al. Ann Oncol. 2001 12(11): 1651-2). Further, it has been reported that the efficacy and safety of gemcitabine is more dependent on the schedule than on the dosage (Vermorken et al. Br J Cancer 1997 76(11):1489-93).

Although gemcitabine has been developed as an anticancer agent, there has been little serious investigation of gemcitabine as an antiviral agent for two reasons (1) those familiar with gemcitabine as an antitumor agent know that it is so toxic that it is usually be administered only according to a regimen of typically once a week for three to four weeks followed by a “rest week” (see Table 1 below); and (ii) standard antiviral therapy consists of daily administration of nucleoside analogues for an indefinite period, and perhaps for the life of the patient (see Table 2).

TABLE 1
Standard Anticancer Dosages for Gemcitabine

| CANCER | INDICATIONS | DOSE REGIMEN | ADVERSE EFFECTS |
|---|---|---|---|
| Non-Small Cell | For use in combination | 28 day cycle: | Thrombocytopenia, |
| Lung Cancer | with cisplatin for the | gemcitabine (1250 | anemia, rash, |
| | first-line treatment of | mg/m², on days 1, 8 | vomiting, flu-like |
| | patients with | and 15) + cisplatin | syndrome, fevers |
| | inoperable, locally | (100 mg/m² on day | |
| | advanced (Stage IIIA | 1) | |
| | or IIIB) or metastatic | | |
| | (Stage IV) non-small | | |
| | cell lung cancer. | | |
| Pancreatic | Treatment of patients | 1000 mg/m² over 30 | Thrombocytopenia, |
| Cancer | with locally advanced | minutes once weekly | anemia, rash, |
| | (nonresectable stage II | for up to 7 weeks | vomiting, flu-like |
| | or III) or metastatic | | syndrome, fevers |
| | (stage IV) | | |
| | adenocarcinoma of the | | |
| | pancreas. Indicated for | | |
| | first-line treatment and | | |
| | for patients previously | | |
| | treated with a 5- | | |
| | fluorouracil-containing | | |
| | regimen. | | |
| Bladder Cancer | | The recommended | Thrombocytopenia, |
| | | dose for gemcitabine | anemia, rash, |
| | | is 800-1000 mg/m², | vomiting, flu-like |
| | | given by 30 minute | syndrome, fevers |
| | | infusion. The dose | |
| | | should be given on | |
| | | Days 1, 8, and 15 | |
| | | followed by 1 week | |
| | | rest. Optionally, | |
| | | Cisplatin is given at a | |
| | | dose of 70 mg/m² on | |
| | | Day 2 of each 28 day | |
| | | cycle. | |

TABLE 2
Standard Antiviral Dosages for Nucleoside Analogues

| Nucleoside Reverse Transcriptase Inhibitors | | | | | | | |
|---|---|---|---|---|---|---|---|
| | | | | Parent | | | |
| | | | | drug | | | |
| | | | | plasma- | NTP | EC₅₀(μM) | |
| | | Impact of | | serum | intracel- | | T- |
| | | monother- | Adverse | half-life | lular half- | | cell |
| Formula and Name | Dosing* | apy* | events* | (hr)* | life (hr) | PBMC | lines |
| 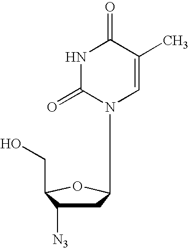 | 200 mg tid(6 pills per day) or 250 mg bid(2 pills per day) | One log decrease in HIV-1 RNA for six months to one year 1 | Nausea, vomiting, headache, neutropenia, anemia, insomnia | 0.8-1.9 | 3-4 | 0.004-0.025 | 0.005-0.006 |
| Zidovudine | | | | | | | |
| [ZDV; AZT; | | | | | | | |
| azidothymidine; 1- | | | | | | | |
| (3′Azido-2′-deoxyribosyl) | | | | | | | |
| thymine; Retrovir ®) | | | | | | | |
| 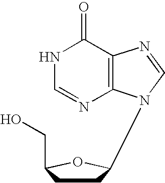 | 400 mg (twice daily for patients ≧60 kg) 250 mg (twice daily for patients <60 kg) | ˜0.8 log decrease in HIV-1 RNA for six months to one year | Diarrhea, nausea, vomiting, peripheral neuropathy, pancreatitis | 0.6-2.7 | 25-40 | 0.01 | |
| #0.002-0.02 | | | | | | | |
| Didanosine | | | | | | | |
| [ddI; 2′,3′- | | | | | | | |
| dideoxyinosine; Videx ®) | | | | | | | |
| 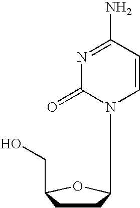 | 0.75 mg tid(2 pills per day) | Less effective than either ddI or AZT | Peripheral neuropathy, mouth ulcers | 1.0-3.0 | 2.6 | 0.002-0.16 | 0.03-0.05 |
| Zalcitabine | | | | | | | |
| [ddC; 2′,3′- | | | | | | | |
| dideoxycytidine; Hivid ®) | | | | | | | |
| 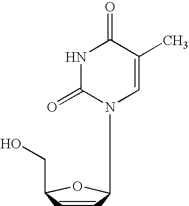 | 40 mg (twice daily for patients ≧60 kg) 30 mg (twice daily for patients <60 kg) | ˜0.8 log decrease in HIV-1 RNA for six months to one year | Peripheral neuropathy | 1.0-1.6 | 3.5 | 0.009 | 0.001-25 |
| Stavudine | | | | | | | |
| [d4T; 3′-deoxy-2′,3′- | | | | | | | |
| didehycrothymidine; | | | | | | | |
| Zerit ®) | | | | | | | |
| 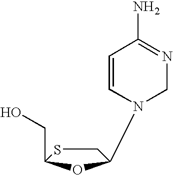 | 150 mg bid or 300 mg qd (approved October 2002) | Limited monothera- py data available | Nausea, headache, malaise, fatigue, diarrhea, cough | 5.0-7.0 | 10.5-15.5 | 0.02-0.395 | 0.07-3.2 |
| Lamivudine | | | | | | | |
| [3TC; (−)-2′,3′-dideoxy- | | | | | | | |
| 3′-thiacytidine; Epivir ®) | | | | | | | |
| 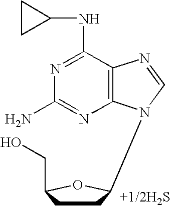 | 300 mg bid, (2 pills per day) | Approximate 1.8 log reduction in HIV-1 RNA at four weeks | Nausea, vomiting, headache, (hypersensi- tivity reaction) | 1.0-2.0 | 3.3 | 0.26-0.23 | 4.1 |
| Abacavir | | | | | | | |
| [ABC; TBC; Ziagen ®) | | | | | | | |
| 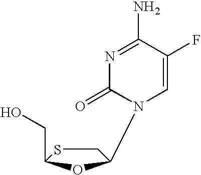 | NDA submitted | ˜2 log reduction at 14 days | Anemia | 1.0-4.0 | ND | 0.0007-0.01 | 0.009-0.5 |
| Emtricitabine | | | | | | | |
| [FTC; Coviracil ®) | | | | | | | |
| 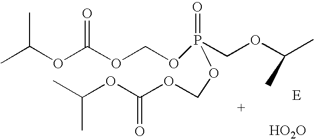 | 300 mg once daily(1 pill per day) | ˜1.2 log decrease in HIV-1 RNA(300 mg) at 28 days | Nausea, headache, asthenia, fatigue, diarrhea, vomiting, pharyngigtis, rash, cough, pain, rhinitis | 17.0 | 10-50 | 0.03 | 0.04-8.5 |
| Tenofovir disoproxil | | | | | | | |
| fumarate | | | | | | | |
| [TDF; bis(POC)PMPA | | | | | | | |
| bix(isopropyloxymethyl | | | | | | | |
| carbonyl)9-R-(2- | | | | | | | |
| phosphonomethoxypropyl) | | | | | | | |
| adenine, a proding of | | | | | | | |
| PMPA] | | | | | | | |

A careful review of Table 2 indicates that antiviral therapy requires daily dosing over a long period of time to sustain a 1-2 log drop in viral load. It has been generally accepted by virologists that if therapy with antiviral drugs is stopped (or administered on an infrequent periodic basis) and virus has not been eliminated, the viral load will rebound quickly, and no sustained therapeutic effect will be achieved.

In 1986, Delong et al from Lilly Research Laboratories published the following abstract.

Synthesis of a group of nucleosides containing 2′,2′-Difluoro-2′-deoxyribose allowed us to examine their antiviral activity. Of particular interest was the cytidine analog which possessed very high in vitro activity against both RNA and DNA viruses without exhibiting toxicity in preformed monolayers. This compound also inhibited HSV-1 mutants resistant to FMAU and acycloguanosine that were thymidine kinase negative and with altered DNA polymerases. Toxicity was observed in rapidly growing cells in culture.

The compound was tested in a variety of animal models for an antiviral effect. Although the compound inhibited virus multiplication in acute virus infections in animals, we were unsuccessful in separating toxicity from virus activity. However, we obtained very high activity in friend leukemia virus infections in mice that could be separated from toxicity by altering the dose schedule. Both spleen enlargement and polyerythroblastosis could be inhibited by 90% under conditions that allowed normal weight gain. A dose schedule calling for treatment every fifth day was possible. Activity was observed by both the oral and Ip routes. Studies were made which indicated that treatment could cause spleen size regression in mice that had enlarged spleens due to the infection.

Emphasis added. Abstracts of the Annual Meeting of the American Society for Microbiology (1986; Abstract No. T-56). While the authors did report an ability to separate activity from toxicity in the case of one viral infection (friend leukemia virus), this was apparently an isolated exception to the reported pattern of demonstrated inability to separate toxicity from activity.

U.S. Pat. No. 5,015,743 discloses a genus of 2,2-difluoro-2-desoxycarbohydrate nucleosides, which includes gemcitabine, for the treatment of viral disorders. The patent teaches that “The antiviral nucleosides of the present invention are used for the treatment of viral infections in the manner usual in the treatment of such pathologies.” In fact, it is now known that gemcitabine cannot be administered indefinitely on a daily basis in accordance with standard antiviral therapy. The patent includes one example of in vitro biological activity, “Test 1” in which the tested compound is not clearly identified. No in vivo data evaluating the toxicity was presented.

WO 02/18404 and US 2003/0008841 A1 filed by Hoffmann-La Roche, Inc. describe certain nucleoside derivatives for the treatment of hepatitis C. Gemcitabine is Compound 243 in. Table 1 of the application, and Example 243. With regard to dosing, the Roche specification teaches that:

The amount of the compound of formula I required for the treatment of hepatitis C virus infections will depend on a number of factors including the severity of the disease and the identity, sex and weight of the recipient and will ultimately be at the discretion of the attendant physician. In general, however, a suitable effective dose is in the range of 0.05 to 100 mg per kilogram of body weight of the recipient per day, preferably in the range 0.1 to 50 mg per kilogram of body weight per day and most preferably in the range of 0.5 to 20 mg of body weight per day. An optimum dose is about 2 to 16 mg per kilogram body weight per day. The desired dose is preferably presented as two, three, four, five, six or more sub-doses administered at appropriate intervals throughout the day. These sub-doses may be administered in unit dosage forms, for example, containing from 1 to 1500 mg, preferably from 5 to 1000 mg, most preferably from 10 to 700 mg of active ingredient per unit dosage form.

Again, the public is taught that it has to use these compounds, including gemcitabine on a daily basis, if not several times a day, to treat the viral infection. Because of the documented toxicity, this teaching at least with regard to gemcitabine appears to fall within the old adage that “dead cells don't contain live virus.” No reasonable physician, however, would kill or seriously damage a patient via chronic drug toxicity as a means to eliminate a viral infection.

Therefore, regardless of these prior reports, no one has seriously considered the real world use of gemcitabine to treat a Flaviviridae infection, including HCV.

U.S. patent application no. 2002/0052317 and WO 02/10743 Al disclose the use of erythropoietin to improve the tolerance to interferon, and which therapy may optionally also include the administration of one of a generic class of nucleoside analogs, including gemcitabine.

Flaviviridae Viruses, Including Hepatitis C Virus

The Flaviviridae is a group of positive single-stranded RNA viruses with a genome size from 9-15 kb. They are enveloped viruses of approximately 40-50 nm. An overview of the Flaviviridae taxonomy is available from the International Committee for Taxonomy of Viruses. The Flaviviridae consists of three genera.

1. Flaviviruses. This genus includes the Dengue virus group (Dengue virus, Dengue virus type 1, Dengue virus type 2, Dengue virus type 3, Dengue virus type 4), the Japanese encephalitis virus group (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), the Modoc virus group, the R¹⁰ Bravo virus group (Apoi virus, R¹⁰ Brovo virus, Saboya virus), the Ntaya virus group, the Tick-Borne encephalitis group (tick born encephalitis virus), the Tyuleniy virus group, Uganda S virus group and the Yellow Fever virus group. Apart from these major groups, there are some additional Flaviviruses that are unclassified.

2. Pestiviruses. This genus includes Bovine Viral Diarrhea Virus-2 (BVDV-2), Pestivirus type 1 (including BVDV), Pestivirus type 2 (including Hog Cholera Virus) and Pestivirus type 3 (including Border Disease Virus).

3. Hepaciviruses. This genus contains only one species, the Hepatitis C virus (HCV), which is composed of many clades, types and subtypes.

HCV was not characterized until 1989 and had previously been referred to as non-A, non-B hepatitis. HCV, in combination with hepatitis B, accounts for 75% of all cases of liver disease worldwide. (Helbling, B., et al., Interferon And Amantadine In Naive Chronic Hepatitis C: A Doubleblind, Randomized, Placebo-Controlled Trial. Hepatology, 2002. 35(2):447-54). Liver failure related to HCV infection is the leading cause of liver transplants in the United States. Since HCV infection is typically mild in its early stages, it is rarely diagnosed until its chronic stages; therefore, HCV is often referred to as the “silent epidemic”. The typical cycle of HCV from infection to symptomatic liver disease takes approximately 20 years, thus the true impact of this disease on the growing infected population will not be apparent for many years. HCV is spread by contact with the blood of an infected person. Individuals with the highest risk factors for HCV infection include:

users of injectable illegal drugs

recipients of blood transfusions or solid organ transplant recipients prior to 1992

recipients of a blood product for clotting problems before 1987

patients on long-term kidney dialysis

individuals that exhibit evidence of liver disease (e.g., persistently abnormal ALT levels)

It is estimated that approximately 4 million people in the United States are infected with HCV, and more than 200 million persons are infected worldwide. (Hewitt, S. E., Recommendations for Prevention and Control of Hepatitis C Virus (HCV) Infection and HCV-related Disease. 1998, Centers for Disease Control and Prevention). During the 1980's an average of 230,000 new infections occurred each year. After 1989, the number of newly infected individuals declined by >80% to 36,000 by 1986. Most of these persons are chronically infected and may be unaware of their infection because they remain asymptomatic. Thus, HCV-related liver disease is potentially one of the greatest public health threats to be faced in this century. Chronic liver disease is the 10th leading cause of death among adults in the United States and accounts for 25,000 deaths annually. Population-based studies estimate that 40% of chronic liver disease is HCV-related. Since most HCV-infected persons are 30-49 years old, the number of deaths associated with HCV-related chronic liver disease may increase substantially over the next 10-20 years. This is not trivial since current medical cost for treating HCV-related complications are estimated to be>600 million dollars annually.

HCV is an RNA virus and this means that it mutates frequently. (www.epidemic.org/index2.html, The Facts about Hepatitis C. 1998, Dartmouth College). Once an infection occurs, HCV creates different genetic variations of itself within the body of the host. The mutated forms frequently differ from their precursors so the immune system cannot recognize them. Thus, even if the immune system succeeds against one variation, the mutant strains quickly take over and become predominate strains. This explains why >80% of individuals infected with HCV will progress to chronic liver disease. HCV has six major genotypes and more than 50 subtypes. In the United States among patients infected with HCV approximately 70% have genotype 1, 15% have genotype 2, and 10% have genotype 3. (McHutchison, J. G., et al., Interferon Alfa-2b Alone Or In Combination With Ribavirin As Initial Treatment For Chronic Hepatitis C. Hepatitis Interventional Therapy Group. N Engl J Med, 1998. 339(21):1485-92). Antiviral therapy is recommended for patients with chronic HCV infection who are at risk for progression to cirrhosis. (Herrine, S. K., Approach To The Patient With Chronic Hepatitis C Virus Infection. Ann Intern Med, 2002. 136(10):747-57). These persons include anti-HCV-antibody positive patients with persistently elevated ALT levels, detectable HCV RNA, and a liver biopsy that indicates either portal or bridging fibrosis or at least moderate inflammation or necrosis. Therapy for HCV is rapidly changing and combination therapy with interferon and ribavirin, a nucleoside analog, is approved in the United States for treatment naive patients with chronic HCV infection. (Hewitt, S. E., Recommendations for Prevention and Control of Hepatitis C Virus (HCV) Infection and HCV-related Disease. 1998, Centers for Disease Control and Prevention). Sustained response rates have been achieved in 40-50% of patients treated with ribavirin plus interferon compared to 15-25% with interferon alone. However, combination therapy in patients with genotype 1, the most prevalent HCV genotype in the United States, is not very successful and sustained response rates among these patients are still <30%. Furthermore, treatment-related side effects often lead to reductions in dose or discontinuation of treatment. Side effects frequently associated with interferon plus ribavirin therapy include, flu-like symptoms, irritability, depression, anemia, bone marrow suppression and renal failure. Ribavirin is teratogenic and contraindicated in women of child-bearing potential.

Due to the public health threat posed by chronic HCV infection and the limitations of current treatments, there is a growing need for innovative therapeutic approaches to treat HCV infection.

Therefore, an object of the present invention is to provide new compositions and methods for the treatment of Flaviviridae, and in particular HCV infection.

### SUMMARY OF INVENTION

It has been surprisingly discovered that a minimal dose of gemcitabine (or its salt, prodrug or derivative, as described herein) can decrease the viral load of hepatitis C in a human patient by up to 2 logs or more in less than several days, and in fact, in certain cases, in 1-2 days or less. This observed rapid and large drop in viral load runs counter to conventional antiviral experience, wherein a drop of 1-2 logs is only stably achieved after approximately 14 days or more of daily sustained therapy. The unexpectedly robust and unique anti-HCV activity of gemcitabine or is salt or prodrug in a human provides the basis for a fundamental shift in the paradigm of antiviral drug dosing, and allows for the first time the conservative and appropriate use of the drug for such treatment.

Therefore, in a first embodiment, the invention provides a method and composition for the treatment of a Flaviviridae infection, and in particular, a hepatitis C viral infection, that includes administering gemcitabine (or its salt, prodrug or derivative, as described herein) in a dosage range of approximately 50 mg/m² to about 1300 mg/ m² per day for one, two or three days, followed by cessation of therapy. Viral load is then optionally monitored over time to evaluate viral rebound. Therapy is not resumed unless a significant viral load is again observed, and then therapy for 1, 2 or 3 days is repeated. This therapy can be continued indefinitely to monitor and maintain the health of the patient.

Flaviviridae viruses that can be treated include all members of the Hepacivirus genus (HCV), Pestivirus genus (BVDV, CSFV, BDV), and the Flavivirus genus (Dengue virus, Japanese encephalitis virus group (including West Nile Virus), and Yellow Fever virus).

In an alternative embodiment, for more severe Flaviviridae infections, gemcitabine (or its salt, prodrug or derivative, as described herein) is administered in a dosage range of approximately 50 mg/m² to about 1300 mg/ m² per day for between one and seven days (e.g. 1, 2, 3, 4, 5, 6, or 7 days) followed by cessation of therapy. Viral load is optionally monitored over time, and after cessation, viral rebound is monitored. Therapy is not resumed unless a significant viral load is again observed, and then therapy for 1-7 days (e.g., independently 1, 2, 3, 4, 5, 6 or 7 days) and more preferred, 1, 2 or 3 days, is repeated. This therapy can be continued indefinitely to monitor the and maintain the health of the patient.

For the first time, this invention discloses that antiviral therapy with gemcitabine or its salt or prodrug can be achieved using an anti-tumor dosing schedule. In certain embodiments, any approved anti-tumor dosage scheduling for gemcitabine can be used to treat a Flaviviridae infection.

In various illustrative and nonlimiting embodiments, the daily dosage of gemcitabine can range from 100-1500 mg per day, alternatively between 200-1000 mg per day, and more particularly between 300-800 mg per day.

In one illustrative embodiment, on Day 1, the patient is dosed via an intravenous infusion and then asked to remain at the clinic for several hours, up to perhaps 12 hours following administration of the dose of medication. The patient is monitored for safety and tolerance, and blood samples taken to measure HCV-RNA pre-dose, and then at 6 hours and 12 hours post-dose. On Day 2, the patient returns to the clinic for safety assessment and viral load measurements. Optional therapy is continued on days 2, 3, 4, 5, 6 and 7. Therapy is then ceased, and the patient is asked to return to the clinic periodically follow up safety and viral load testing.

It is preferred that gemcitabine be administered in the form of an intravenous infusion, because it is known that gemcitabine is rapidly converted to its uracil derivative in the digestive tract. If it is preferred to administer gemcitabine orally, then the compound should preferably be administered in the form of a prodrug that protects the cytosinyl amine group from rapid deamination without causing an adverse effect on activity. Nonlimiting methods to increase the half-life of the cytosine base in vivo include administering the compound in the N-acylated, N-alkylated or N-arylated form.

Prodrugs also include amino acid derivatives on either the hydroxyl or amino functions to create esters and amides of the disclosed nucleosides (see, e.g., European Patent Specification No. 99493, which describes amino acid esters of acyclovir, specifically the glycine and alanine esters which show improved water-solubility compared with acyclovir itself, and U.S. Pat. No. 4,957,924 (Beauchamp), which discloses the valine ester of acyclovir, characterized by side-chain branching adjacent to the α-carbon atom, which showed improved bioavailability after oral administration compared with the alanine and glycine esters). A process for preparing such amino acid esters is disclosed in U.S. Pat. No. 4,957,924 (Beauchamp). As an alternative to the use of valine itself, a functional equivalent of the amino acid may be used (e.g., an acid halide such as the acid chloride, or an acid anhydride). In such a case, to avoid undesirable side-reactions, it may be is advantageous to use an amino-protected derivative.

As an example of the invention, a male patient exhibiting multifocal HCC, cirrhosis, and ischaemic hepatitis infected with HCV was administered 1200 mg gemcitabine HCl in 1000 minutes associated with oxaliplatine. The tolerance was acceptable, and thus the next day the patient was given a second dosage of approximately 700 mg of gemcitabine. Before the second dosage the baseline viral load was 6.49 log copies/mL. The second perfusion of gemcitabine was stopped after approximately 700 mg because of heart problems, which were apparently unrelated to the gemcitabine therapy. The HCV RNA measurement eight hours after the second dosage was 4.04 log copies/mL, indicating an approximate 2.5 log drop in eight hours.

In an alternative embodiment, gemcitabine or its salt, prodrug or derivative is administered according to the regimen described herein in combination or alternation with one or more other anti-Flaviviridae active agents. The other active agents (as described-in more detail below) are administered in a manner that maximizes their effectiveness in combination with this regimen.

### BRIEF DESCRIPTION OF THE FIGURES

FIG. 1 is an illustration of the self-potentiating actions of gemcitabine and DNA repair.

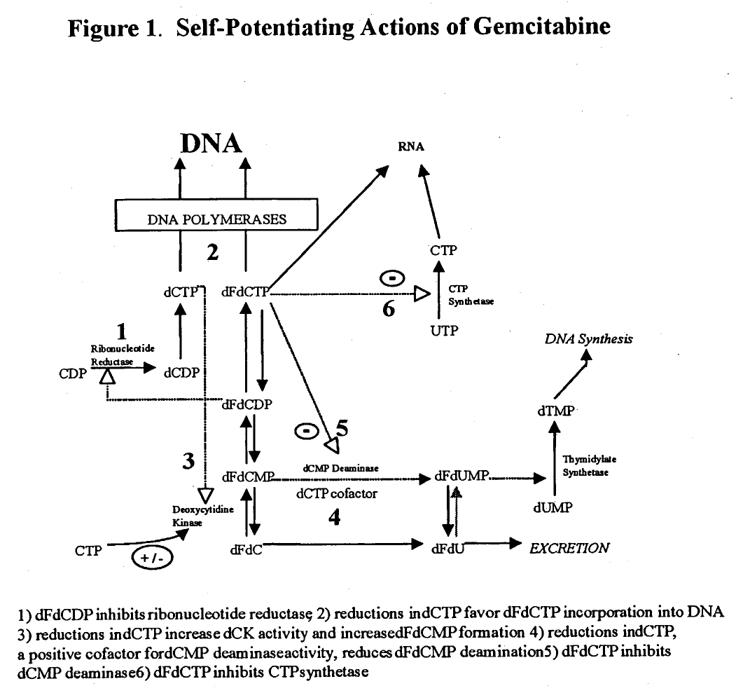

FIG. 2 is a graphical depiction of the dose-dependant reduction of the replicon HCV RNA based on treatment with Gemcitabine (♦: ΔCt for HCV RNA). This viral reduction was compared to the reduction of cellular DNA levels (ribosomal DNA) or cellular RNA levels (ribosomal RNA) to obtain the therapeutic index ΔCt values (▴: HCV-rDNA ΔΔCt; X: HCV-rRNA ΔCt).

### DETAILED DESCRIPTION OF THE INVENTION

It has been surprisingly discovered that a minimal dose of gemcitabine can decrease the viral load of hepatitis C in a human patient by up to 2 logs or more in less than several days, and in fact, in certain cases, in 1-2 days or less. This observed rapid and large drop in viral load runs counter to conventional antiviral experience, wherein a drop of 1-2 logs is only stably achieved after approximately 14 days or more of daily sustained therapy. The unexpectedly robust and unique anti-HCV activity of gemcitabine in a human provides the basis for a fundamental shift in the paradigm of antiviral drug dosing, and allows for the first time the conservative and appropriate use of the drug for such treatment.

Therefore, in a first embodiment, the invention provides a method and composition for the treatment of a Flaviviridae infection, and in particular, a hepatitis C viral infection, that includes administering gemcitabine or its pharmaceutically acceptable salt or prodrug in a dosage range of approximately 50 mg/m² to about 1300 mg/ m² per day for one, two or three days, followed by cessation of therapy. Viral load is then optionally monitored over time to evaluate viral rebound. Therapy is not resumed unless a significant viral load is again observed, and then therapy for 1, 2 or 3 days is repeated. This therapy can be continued indefinitely to monitor the and maintain the health of the patient.

Flaviviridae viruses that can be treated include all members of the Hepacivirus genus (HCV), Pestivirus genus (BVDV, CSFV, BDV), and the Flavivirus genus (Dengue virus, Japanese encephalitis virus group (including West Nile Virus), and Yellow Fever virus).

In an alternative embodiment, for more severe Flaviviridae infections, gemcitabine or its pharmaceutically acceptable salt or prodrug is administered in a dosage range of approximately 50 mg/m² to about 1300 mg/ m² per day for between one and seven days, followed by cessation of therapy. Viral load is then optionally monitored over time to evaluate viral rebound. Therapy is not resumed unless a significant viral load is again observed, and then therapy for 1-7 days, and more preferred, 1, 2 or 3 days, is repeated. This therapy can be continued indefinitely to monitor and maintain the health of the patient.

I. Compounds of the Invention

In a particular embodiment of the present invention, a β-D nucleoside of the formula:

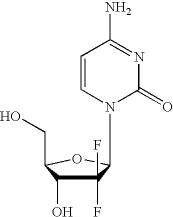

its β-L enantiomer, or its pharmaceutically acceptable salt or prodrug, is provided for the treatment or prophylaxis of a Flaviviridae infection, and in particular HCV. In a preferred embodiment, the compound is gemcitabine or its pharmaceutically acceptable salt, ester or prodrug. The compound, by way of example, can be alkylated, acylated, or otherwise derivatized at the N⁴ and/or 3′ and/or 5′-position to modify its activity, bioavailability, stability or otherwise alter the properties of the nucleoside. This may make it more stable for non-intravenous formulations. In one embodiment, the compound is acylated at the N⁴ and/or 3′ and/or 5′ position with an amino acid, such as valine.

In a broader aspect of the invention, the active compound is a β-D or β-L nucleoside of the general formula (I): or its pharmaceutically acceptable salt or prodrug thereof (referred to herein as a gemcitabine derivative) wherein:

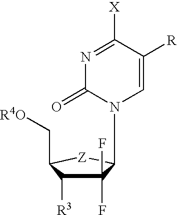

R is H, halogen (F. Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR₁₂, CH═CHCO₂H, CH═CHCO₂R′;

X is H, halogen, OH, OR′, OCH₃, SH, SR′, SCH₃, NH₂, NHR′, NR₁₂, CH₃;

each R′ is independently a hydrogen, lower alkyl of C₁-C₆ or lower cycloalkyl of C₁-C₆;

Z is O, S or CH₂;

R⁴ is H, mono-phosphate, di-phosphate, tri-phosphate; a stabilized phosphate prodrug; acyl; alkyl; sulfonate ester; a lipid, a phospholipid; an amino acid; a carbohydrate; a peptide; a cholesterol; or other pharmaceutically acceptable leaving group which when administered in vivo is capable of providing a compound wherein R⁴ is H or phosphate; and

R³ is F or OH.

In another embodiment of the present invention, the active compound is a β-D or β-L nucleoside of the general formula (I), wherein X is NH₂ and R is halogen or its pharmaceutically acceptable salt or prodrug thereof or its use as further described herein is provided.

In another embodiment of the present invention, the active compound is a β-D or β-L nucleoside of the general formula (I), wherein X is NH₂ and R is alkyl or its pharmaceutically acceptable salt or prodrug thereof or its use as further described herein is provided. In another embodiment of the present invention, the active compound is a β-D or β-L nucleoside of the general formula (I), wherein X is NH₂ and R is halogenated alkyl or its pharmaceutically acceptable salt or prodrug thereof or its use as further described herein is provided.

In another embodiment of the present invention, the active compound is a β-D or β-L nucleoside of the general formula (I), wherein X is CH₃ and R is NH₂ or its pharmaceutically acceptable salt or prodrug thereof or its use as further described herein is provided.

In another embodiment of the present invention, the active compound is a β-D or β-L nucleoside of the general formula (I), wherein X is OR′ and R is halogen or its pharmaceutically acceptable salt or prodrug thereof or its use as further described herein is provided.

In another embodiment of the present invention, the active compound is a β-D or β-L nucleoside of the general formula (I), wherein X is NH₂, R is halogen, R⁴ is hydrogen or its pharmaceutically acceptable salt or prodrug thereof or its use as further described herein is provided.

In another embodiment of the present invention, the active compound is a β-D or β-L nucleoside of the general formula (I), wherein X is NH₂ or its pharmaceutically acceptable salt or prodrug thereof or its use as further described herein is provided. In another embodiment of the present invention, the active compound is a β-D or β-L nucleoside of the general formula (I), wherein X is NH₂ and R is alkenyl or its pharmaceutically acceptable salt or prodrug thereof or its use as further described herein is provided.

In another embodiment of the present invention, the active compound is a β-D or β-L nucleoside of the general formula (I), wherein X is NH₂ and R is alkynl or its pharmaceutically acceptable salt or prodrug thereof or its use as further described herein is provided.

In another embodiment of the present invention, the active compound is a β-D or β-L nucleoside of the general formula (1), wherein X is NH₂ and R is halogenated alkenyl or its pharmaceutically acceptable salt or prodrug thereof or its use as further described herein is provided.

In another embodiment of the present invention, the active compound is a β-D or β-L nucleoside of the general formula (I), wherein X is NH₂ and R is halogen alkynyl or its pharmaceutically acceptable salt or prodrug thereof or its use as further described herein is provided.

In another embodiment of the present invention, the active compound is a β-D or β-L nucleoside of the general formula (I), wherein X is NH₂ and R is alkoxy or its pharmaceutically acceptable salt or prodrug thereof or its use as further described herein is provided.

In another embodiment of the present invention, the active compound is a β-D or β-L nucleoside of the general formula (I), wherein X is NH₂ and R is CO₂H or its pharmaceutically acceptable salt or prodrug thereof or its use as further described herein is provided.

In another embodiment of the present invention, the active compound is a β-D or β-L nucleoside of the general formula (I), wherein X is NH₂ and R is CO₂R′ or its pharmaceutically acceptable salt or prodrug thereof or its use as further described herein is provided.

In another embodiment of the present invention, the active compound is a β-D or β-L nucleoside of the general formula (I), wherein X is NH₂ and R is CONH₂ or its pharmaceutically acceptable salt or prodrug thereof or its use as further described herein is provided.

In another embodiment of the present invention, the active compound is a β-D or β-L nucleoside of the general formula (I), wherein X is NH₂ and R is CONHR′ or its pharmaceutically acceptable salt or prodrug thereof or its use as further described herein is provided.

In another embodiment of the present invention, the active compound is a β-D or β-L nucleoside of the general formula (I), wherein X is NH₂ and R is halogen or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is halogen or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is alkyl or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is halogenated alkyl or its pharmaceutically acceptable salt or prodrug thereof or its use as further described herein is provided.

In another embodiment of the present invention, the active compound is a β-D or β-L nucleoside of the general formula (I), wherein X is CH₃, R³ is OH, and R is NH₂ or its pharmaceutically acceptable salt or prodrug thereof or its use as further described herein is provided.

In another embodiment of the present invention, the active compound is a β-D or β-L nucleoside of the general formula (I), wherein X is OR′, R³ is OH, and R is halogen or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, R is halogen, R⁴ is hydrogen or its pharmaceutically acceptable salt or prodrug thereof or its use as further described herein is provided.

In another embodiment of the present invention, the active compound is a β-D or β-L nucleoside of the general formula (I), wherein X is NH₂ and R³ is OH, or its pharmaceutically acceptable salt or prodrug thereof or its use as further described herein is provided.

In another embodiment of the present invention, the active compound is a β-D or β-L nucleoside of the general formula (I), wherein X is CH₃, R³ is F, and R is alkenyl or its pharmaceutically acceptable salt or prodrug thereof or its use as further described herein is provided.

In another embodiment of the present invention, the active compound is a β-D or β-L nucleoside of the general formula (I), wherein X is CH₃, R³ is OH, and R is alkynl or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is halogenated alkenyl or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is halogen alkynyl or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is alkoxy or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is CO₂H or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is CO₂R′ or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is CONH₂ or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is CONHR′ or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is halogen or its pharmaceutically acceptable salt or prodrug thereof or its use as further described herein is provided.

In another embodiment of the present invention, the active compound is a β-D or β-L nucleoside of the general formula (I), wherein X is NH₂, R³ is OH, and R is halogen, and R⁴ is H or its pharmaceutically acceptable salt or prodrug thereof or its use as further described herein is provided.

In another embodiment of the present invention, the active compound is a β-D or β-L nucleoside of the general formula (I), wherein X is SH₂, R³ is OH, and R is halogen or its pharmaceutically acceptable salt or prodrug thereof or its use as further described herein is provided.

In another embodiment of the present invention, the active compound is a β-D or β-L nucleoside of the general formula (I), wherein X is NHR¹, R³ is OH, and R is halogen or its pharmaceutically acceptable salt or prodrug thereof or its use as further described herein is provided.

In another embodiment of the present invention, the active compound is a β-D or β-L nucleoside of the general formula (I), wherein R is halogen or its pharmaceutically acceptable salt or prodrug thereof or its use as further described herein is provided.

In another embodiment of the present invention, the active compound is a β-D or β-L nucleoside of the general formula (I), wherein R is alkyl or its pharmaceutically acceptable salt or prodrug thereof or its use as further described herein is provided.

In another embodiment of the present invention, the active compound is a β-D or β-L nucleoside of the general formula (I), wherein R is alkenyl or its pharmaceutically acceptable salt or prodrug thereof or its use as further described herein is provided.

In another embodiment of the present invention, the active compound is a β-D or β- L nucleoside of the general formula (I), wherein R is alkynyl or its pharmaceutically acceptable salt or prodrug thereof or its use as further described herein is provided.

In another embodiment of the present invention, the active compound is a β-D or β-L nucleoside of the general formula (I), wherein R is halogenated alkenyl or its pharmaceutically acceptable salt or prodrug thereof or its use as further described herein is provided.

In another embodiment of the present invention, the active compound is a β-D or β-L nucleoside of the general formula (I), wherein R is halogenated alkynl or its pharmaceutically acceptable salt or prodrug thereof or its use as further described herein is provided.

In another embodiment of the present invention, the active compound is a β-D or β-L nucleoside of the general formula (I), wherein R is alkoxy or its pharmaceutically acceptable salt or prodrug thereof or its use as further described herein is provided.

In another embodiment of the present invention, the active compound is a β-D or β-L nucleoside of the general formula (I), wherein R is CO₂H or its pharmaceutically acceptable salt or prodrug thereof or its use as further described herein is provided.

In another embodiment of the present invention, the active compound is a β-D or β-L nucleoside of the general formula (I), wherein X is OR′ or its pharmaceutically acceptable salt or prodrug thereof or its use as further described herein is provided.

In another embodiment of the present invention, the active compound is a β-D or β-L nucleoside of the general formula (I), wherein X is NHR′ or its pharmaceutically acceptable salt or prodrug thereof or its use as further described herein is provided. In another embodiment of the present invention, the active compound is a β-D or β-L nucleoside of the general formula (I), wherein X is CONH₂ or its pharmaceutically acceptable salt or prodrug thereof or its use as further described herein is provided.

In another embodiment of the present invention, the active compound is a β-D or β-L nucleoside of the general formula (I), wherein R³ is F or its pharmaceutically acceptable salt or prodrug thereof ′or its use as further described herein is provided.

In another embodiment of the present invention, the active compound is a β-D or β-L nucleoside of the general formula (I), wherein R³ is OH or its pharmaceutically acceptable salt or prodrug thereof or its use as further described herein is provided.

In another embodiment of the present invention, the active compound is a β-D or β-L nucleoside of the general formula (I), wherein R⁴ is H or its pharmaceutically acceptable salt or prodrug thereof or its use as further described herein is provided.

In another embodiment of the present invention, the active compound is a β-D or β-L nucleoside of the general formula (I), wherein R⁴ is mono-phosphate or its pharmaceutically acceptable salt or prodrug thereof or its use as further described herein is provided.

In another embodiment of the present invention, the active compound is a β-D or β-L nucleoside of the general formula (I), wherein R⁴ is di-phosphate or its pharmaceutically acceptable salt or prodrug thereof or its use as further described herein is provided.

In another embodiment of the present invention, the active compound is a β-D or β-L nucleoside of the general formula (I), wherein R⁴ is tri-phosphate or its pharmaceutically acceptable salt or prodrug thereof or its use as further described herein is provided.

In another embodiment of the present invention, the active compound is a β-D or β-L nucleoside of the general formula (I), wherein R⁴ is acyl or its pharmaceutically acceptable salt or prodrug thereof or its use as further described herein is provided.

In another embodiment of the present invention, the active compound is a β-D or β-L nucleoside of the general formula (I), wherein R⁴ is H and Z is O or its pharmaceutically acceptable salt or prodrug thereof or its use as further described herein is provided.

In another embodiment of the present invention, the active compound is a β-D or β-L nucleoside of the general formula (I), wherein R⁴ is H and Z is CH₂ or its pharmaceutically acceptable salt or prodrug thereof or its use as further described herein is provided. In another embodiment of the present invention, the active compound is a β-D or β-L nucleoside of the general formula (I), wherein R is H, R³ is F, and R⁴ is acyl or its pharmaceutically acceptable salt or prodrug thereof or its use as further described herein is provided.

In another embodiment of the present invention, the active compound is a β-D or β-L nucleoside of the general formula (I), wherein R⁴ is H and R is OR′ or its pharmaceuitcally acceptable salt or prodrug thereof or its use as further described herein is provided.

Definitions

The term “alkyl,” as used herein, unless otherwise specified, refers to a saturated straight, branched, or cyclic, primary, secondary, or tertiary hydrocarbon, including but not limited to those of C₁ to C₁₆, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The alkyl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, azido, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl,” as used herein, and unless otherwise specified, refers to a C₁ to C₄ saturated straight, branched, or if appropriate, a cyclic (for example, cyclopropyl) alkyl group, including both substituted and unsubstituted forms.

The term “alkylene” or “alkenyl” refers to a saturated hydrocarbyldiyl radical of straight or branched configuration, including but not limited to those that have from one to ten carbon atoms. Included within the scope of this term are methylene, 1,2-ethane-diyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, azido, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “aryl,” as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of bromo, chloro, fluoro, iodo, hydroxyl, azido, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term amino acid includes naturally occurring and synthetic α, β γ or δ amino acids, and includes but is not limited to, alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, α-alanyl, P-valinyl, β-leucinyl, β-isoleuccinyl, β-prolinyl, β-phenylalaninyl, β-tryptophanyl, β--methioninyl, β-glycinyl, β-serinyl, β-threoninyl, β-cysteinyl, β--tyrosinyl, β--asparaginyl, β--glutaminyl, β-aspartoyl, β-glutaroyl, β--lysinyl, β--argininyl, and β-histidinyl.

The term “aralkyl,” as used herein, and unless otherwise specified, refers to an aryl group as defined above linked to the molecule through an alkyl group as defined above. The term “alkaryl” or “alkylaryl” as used herein, and unless otherwise specified, refers to an alkyl group as defined above linked to the molecule through an aryl group as defined above. In each of these groups, the alkyl group can be optionally substituted as describe above and the aryl group can be optionally substituted with one or more moieties selected from the group consisting of alkyl, halo, haloalkyl, hydroxyl, carboxyl, acyl, acyloxy, amino, amido, azido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included within the scope of the term aryl are phenyl; naphthyl; phenylmethyl; phenylethyl; 3,4,5-trihydroxyphenyl; 3,4,5-trimethoxyphenyl; 3,4,5-triethoxy-phenyl; 4-chlorophenyl; 4-methylphenyl; 3,5-di-tertiarybutyl-4-hydroxyphenyl; 4-fluorophenyl; 4-chloro-1-naphthyl; 2-methyl-1-naphthylmethyl; 2-naphthylmethyl; 4-chlorophenylmethyl; 4tbutylphenyl; 4-t-butylphenylmethyl and the like.

The term “alkylamino” or “arylamino” refers to an amino group that has one or two alkyl or aryl substituents, respectively.

The term “halogen,” as used herein, includes fluorine, chlorine, bromine and iodine.

The term “enantiomerically enriched” is used throughout the specification to describe a nucleoside which includes at least about 95%, preferably at least 96%, more preferably at least 97%, even more preferably, at least 98%, and even more preferably at least about 99% or more of a single enantiomer of that nucleoside. In a preferred embodiment, the nucleoside analog is provided in enantiomerically enriched form.

The term “host,” as used herein, refers to a unicellular or multicellular organism in which the virus can replicate, including cell lines and animals, and preferably a human.

Alternatively, the host can be carrying a part of the viral genome, whose replication or function can be altered by the compounds of the present invention. The term host specifically refers to infected cells, cells transfected with all or part of the viral genome and animals, in particular, primates (including chimpanzees) and humans. Relative to abnormal cellular proliferation, the term “host” refers to unicellular or multicellular organism in which abnormal cellular proliferation can be mimicked. The term host specifically refers to cells that abnormally proliferate, either from natural or unnatural causes (for example, from genetic mutation or genetic engineering, respectively), and animals, in particular, primates (including chimpanzees) and humans. In most animal applications of the present invention, the host is a human patient. Veterinary applications, in certain indications, however, are clearly anticipated by the present invention (such as bovine viral diarrhea virus in cattle, hog cholera virus in pigs, and border disease virus in sheep).

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The compounds of this invention either possess antiviral activity against Flaviviridae viruses or anti-proliferative activity against abnormal cellular proliferation, or are metabolized to a compound that exhibits such activity.

Stereoisomerism and Polymorphism

Compounds of the present invention have at least two chiral centers, and may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. Optically active forms of the compounds can be prepared using any method known in the art, including by resolution of the racemic form by recrystallization techniques, by synthesis from optically-active starting materials, by chiral synthesis, or by chroma tographic separation using a chiral stationary phase.

Examples of methods to obtain optically active materials include at least the following.

i) Physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) chiral gas chromatogaphy—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier.

Separation occurs as a result of the non-racemic chiral nature of the membrane that allows only one enantiomer of the racemate to pass through.

Chiral chromatography, including simulated moving bed chromatography, is used in one embodiment. A wide variety of chiral stationary phases are commercially available.

Pharmaceutical Compositions

Pharmaceutical compositions based upon a compound of formula (I) or its pharmaceutically acceptable salt or prodrug can be prepared in a therapeutically effective amount for treating a Flaviviridae virus, optionally in combination with a pharmaceutically acceptable additive, carrier or excipient. The therapeutically effective amount may vary with the infection or condition to be treated, its severity, the treatment regimen to be employed, the pharmacokinetics of the agent used, as well as the patient treated. In one aspect according to the present invention, the compound according to the present invention is formulated preferably in admixture with a pharmaceutically acceptable carrier. In general, it is preferable to administer the pharmaceutical composition in an intravenous form, but formulations may be prepared for administration via oral, parenteral, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route. In particular, a modification of a desired compound to render it more soluble in water or other vehicle, for example, may be easily accomplished by routine modification (salt formulation, esterification, etc.).

In certain pharmaceutical dosage forms, for example an oral formuation, the prodrug form of the compound, especially including an acylated (acetylated or other) and ether derivative, phosphate ester or a salt forms of the present compound, is preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound in the treatment of Flaviviridae (including HCV) infections.

The amount of compound included within therapeutically active formulations, according to the present invention, is an effective amount for treating a Flaviviridae (including HCV) infection.

Administration of the active compound may range from continuous (intravenous drip) to several oral administrations (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the severity of disease in the patient.

In a first embodiment, the invention provides a method and composition for the treatment of a Flaviviridae infection, and in particular, a hepatitis C viral infection, that includes administering gemcitabine or its pharmaceutically acceptable salt or prodrug or derivative in a dosage range of approximately 50 mg/m² to about 1300 mg/ m² per day for one, two or three days, followed by cessation of therapy. In an alternative embodiment, for more severe Flaviviridae infections, gemcitabine or its pharmaceutically acceptable salt or prodrug or derivative is administered in a dosage range of approximately 50 mg/m² to about 1300 mg/ m² per day for between one and seven days (e.g., 1, 2, 3, 4, 5, 6, or 7 days), followed by cessation of therapy.

The daily dosage of gemcitabine or another active compound according to the invention can be selected to maximize the therapeutic effect. Examples of nonlimiting dosage ranges are between 100-1500 mg per day, alternatively between 200-1000 mg per day, and more particularly between 300-800 mg per day.

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts as well as hydrochloride and hydrobromide salts.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by refer ence herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., intravenous, or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient. For parenteral formulations, the carrier will usually comprise sterile water or aqueous sodium chloride solution, though other ingredients, including those that aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

In addition, the compounds according to the present invention can be administered in combination or alternation with one or more antiviral, anti-HIV, anti-HBV, anti-HCV or anti-herpetic agent or interferon, anti-cancer or antibacterial agents, including other compound. The preferred compounds include interferon alpha, ribavirin. Certain compounds according to the present invention may be effective for enhancing the biological activity of certain agents according to the present invention by reducing the metabolism, catabolism or inactivation of other compounds and as such, are co-administered for this intended effect.

In an additional embodiment, the method for the treatment or prophylaxis of a mammal having a virus-associated disorder which comprises administering to the mammal a pharmaceutically effective amount of gemcitabine, or its pharmaceutically acceptable salt or prodrug thereof, optionally in a combination or alternation with one or more other anti-virally effective agent(s), optionally in a pharmaceutically acceptable carrier or diluent, as disclosed herein, is provided. In a preferred embodiment, the mammal is a human. In particular, the invention includes methods for treating or preventing and uses for the treatment or prophylaxis of a Flaviviridae infection, including all members of the Hepacivirus genus (HCV), Pestivirus genus (BVDV, CSFV, BDV), or Flavivirus genus (Dengue virus, Japanese encephalitis virus group (including West Nile Virus), and Yellow Fever virus).

This invention is further illustrated in the following sections. The Examples contained therein are set forth to aid in an understanding of the invention. This section is not intended to, and should not be interpreted to, limit in any way the invention set forth in the claims that follow thereafter.

Therapies for the Treatment of Flaviviridae Infection

It has been recognized that drug-resistant variants of viruses can emerge after prolonged treatment with an antiviral agent. Drug resistance most typically occurs by mutation of a gene that encodes for an enzyme used in the viral replication cycle, and most typically in the case of HCV, the RNA-dependent-RNA polymerase. It has been demonstrated that the efficacy of a drug against viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter, of the drug can be altered by such combination or alternation therapy. In general, combination therapy is typically preferred over alternation therapy because it induces multiple simultaneous stresses on the virus. Examples of agents that have been identified as active against Flaviviridae, and in particular the hepatitis C virus, and thus can be used in combination or alternation with gemcitabine, its salt, prodrug or derivative are described in the following numbered paragraphs.

(1) interferon and/or ribavirin.

(2) Substrate-based NS3 protease inhibitors (Attwood et al., Antiviralpeptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734).

(3) Non-substrate-based inhibitors such as 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing apara-phenoxyphenyl group.

(4) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193.

(5) Thiazolidines and benzanilides identified in Kakiuchi N. et al. J. EBS Letters 421, 217-220; Takeshita N. et al. Analytical Biochemistry, 1997, 247, 242-246.

(6) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griscofuluum, which demonstrates activity in a scintillation proximity assay (Chu M. et al., Bioorganic and Medicinal Chemistry Letters 9, 1949-1952).

(7) Selective NS3 inhibitors based on the macromolecule elgin c, isolated from leech (Qasim M. A. et al., Biochemistry, 1997, 36, 1598-1607).

(8) Helicase inhibitors (Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554).

(9) Polymerase inhibitors such as nucleotide analogues, gliotoxin (Ferrari R. et al. Journal of Virology, 1999, 73, 1649-1654), and the natural product cerulenin (Lohlnann V. et al., Virology, 1998, 249, 108-118).

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257).

(11) Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. JP-10101591).

(12) Nuclease-resistant ribozymes (Maccjak, D. J. et al., Hepatology 1999, 30, abstract 995).

(13) Nucleoside analogs have also been developed for the treatment of Flaviviridae infections.

(14) Idenix Pharmaceuticals, Ltd. discloses branched nucleosides, and their use in the treatment of HCV and flaviviruses and pestiviruses in International Publication Nos. WO 01/90121 (filed May 23, 2001) and WO 01/92282 (filed May 26, 2001). A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier.

(15) WO 01/96353 (filed Jun. 15, 2001) to Indenix Pharmaceuticals, Ltd. discloses 3′-prodrugs of 2′-deoxy-β-L-nucleosides for the treatment of HBV. U.S. Pat. No. 4,957,924 to Beauchamp discloses various therapeutic esters of acyclovir.

(16) Other patent applications disclosing the use of certain nucleoside analogs to treat hepatitis C virus include: PCT/CAOO/01316 (WO 01/32153; filed Nov. 3, 2000) and PCT/CAO1/00197 (WO 01/60315; filed Feb. 19, 2001) filed by BioChem Pharma, Inc. (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication No. WO 01/79246 (filed Apr. 13, 2001) and WO 02/32920 (filed Oct. 18, 2001) by Pharmasset.

(17) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.).

(18) Other compounds currently in clinical development for treatment of hepatitis c virus include: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

(19) U.S. Pat. No. 6,348,587 to Emory University and the University of Georgia Research Foundation discloses the use of 2′-fluoronucleosides for the treatment of HIV, hepatitis B, hepatitis C and abnormal cellular proliferation.

Synthetic Protocol For pyrimidine nucleosides, uridine derivative (1, Scheme 1) is the starting material, which is converted into 2,2′-anhydro derivative (2) which is treated with HF in anhydrous dioxane (Codington et al., J. Org. Chem., 1964, 29, 558). The corresponding 2′-fluoro-2′-deoxyuridine derivative (3) is obtained in 40-50% yield. Modification at the 4 position in 3 can be achieved by various methods. 2′-Fluoro-2′-deoxycytidine derivatives (4, R═R′═R″═H) can be readily prepared from 3 by the well-known procedures via thiation or chlorination.

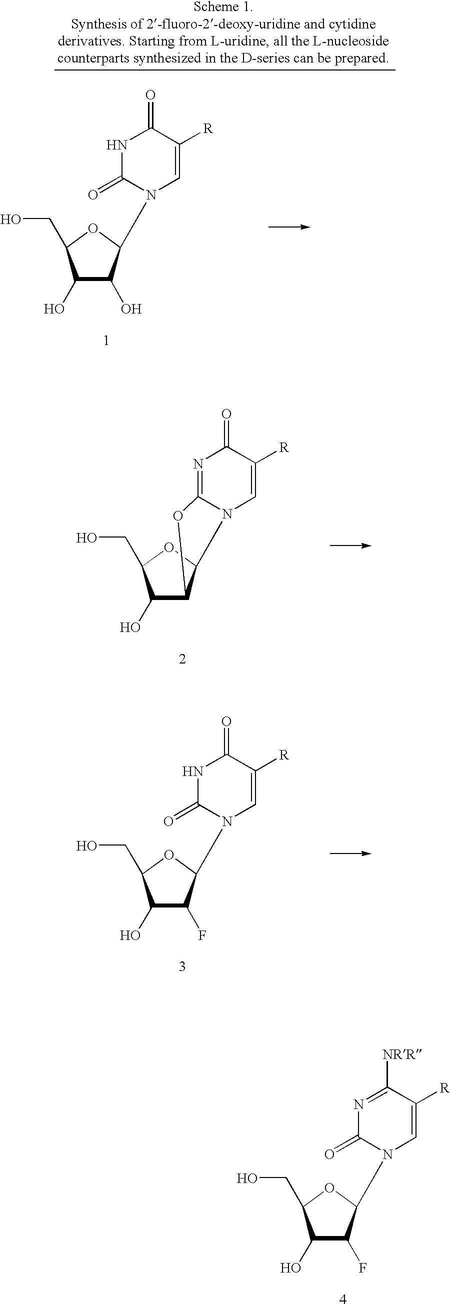

gem-Difluoronucleosides can be obtained by condensation of 2,2-difluoro-1-O-acetyl-3,5-di-O-benzoyl-2-deoxo-D-ribofuranos-2-ulose (8, Scheme 2) with various silyated pyrimidine 20 bases or with purines by the sodium salt method. The sugar can be readily prepared from 2,3-O-isopropylidene-D-glyceral (5) and ethyl bromodifluoroacetate (6) by Reformatzky reaction, followed by acidic removal of protecting groups to give lactone 7. Benzoylation of 7, and subsequent conversion of the lactone to lactol by DIBAL reduction and acetylation affords 8.

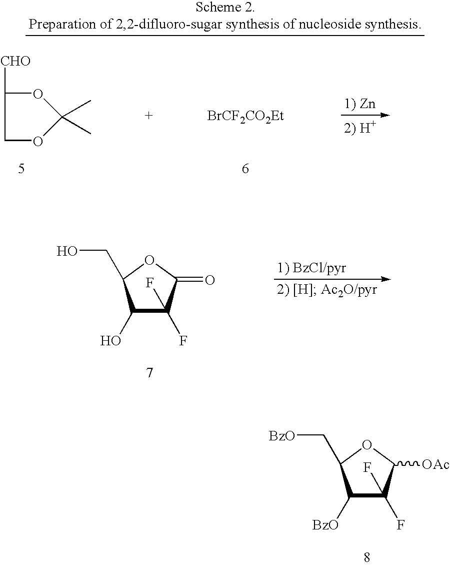

### EXAMPLES

The following working examples provide a further understanding of the method of the present invention. These examples are of illustrative purposes, and are not meant to limit the scope of the invention. Equivalent, similar or suitable solvents, reagents or reaction conditions may be substituted for those particular solvents, reagents or reaction conditions described without departing from the general scope of the method

### Example 1

Antiviral testing of candidate compounds for Flaviviridae: The HCV replicon system in Huh7 cells. Huh7 cells harboring the HCV replicon can be cultivated in DMEM media (high glucose, no pyruvate) containing 10% fetal bovine serum, IX non-essential Amino Acids, Pen-Strep-Glu (100 units/liter, 100 microgram/liter, and 2.92 mg/liter, respectively) and 500 to 1000 microgram/milliliter G418. Antiviral screening assays can be done in the same media without G418 as follows: in order to keep cells in logarithmic growth phase, seed cells in a 96-well plate at low density, for example 1000 cells per well. Add the test compound immediate after seeding the cells and incubate for a period of 3 to 7 days at 37° C. in an incubator. Media is then removed, and the cells are prepared for total nucleic acid extraction (including replicon RNA and host RNA). Replicon RNA can then be amplified in a Q-RT-PCR protocol, and quantified accordingly. The observed differences in quantification of replicon RNA is one way to express the antiviral potency of the test compound. A typical experiment demonstrates that in the negative control and in the non-active compounds-settings a comparable amount of replicon is produced. This can be concluded because the measured threshold-cycle for HCV RT-PCR in both setting is close to each other. In such experiments, one way to express the antiviral effectiveness of a compound is to subtract the threshold RT-PCR cycle of the test compound with the average threshold RT-PCR cycle of the negative control. This value is called DeltaCt (ΔCt or DCt).

A ΔCt of 3.3 equals a 1-log reduction (equals EC₉₀) in replicon production. Compounds that result in a reduction of HCV replicon RNA levels of greater than 2 □Ct values (75% reduction of replicon RNA) are candidate compounds for antiviral therapy. Such candidate compounds are belonging to structures with general formula (I). As a positive control, recombinant interferon alfa-2a (Roferon-A, Hoffmann-Roche, New Jersey, USA) is taken alongside as positive control.

However, this HCV ACt value does not include any specificity parameter for the replicon encoded viral RNA-dependent RNA polymerase. In a typical setting, a compound might reduce both the host RNA polymerase activity and the replicon-encoded polymerase activity. Therefore, quantification of rRNA (or any other host RNA polymerase I product) or beta-actin mRNA (or any other host RNA polymerase II) and comparison with RNA levels of the no-drug control is a relative measurement of the effect of the test compound on host RNA polymerases.

With the availability of both the HCV ΔCt data and the rRNA ΔCt, a specificity parameter can be introduced. This parameter is obtained by subtracting both ΔCt values from each other. This results in Delta-DeltaCT values (ΔCt or DDCt); a value above 0 means that there is more inhibitory effect on the replicon encoded polymerase, a ΔCt value below 0 means that the host rRNA levels are more affected than the replicon levels. As a general rule, ΔCt values above 2 are considered as significantly different from the no-drug treatment control, and hence, exhibits appreciable antiviral activity. However, compounds with a ΔCt value of less than 2, but showing limited molecular cytotoxicty data (rRNA ACT between 0 and 2) are also possible active compounds.

In another typical setting, a compound might reduce the host RNA polymerase activity, but not the host DNA polymerase activity. Therefore, quantification of rDNA or beta-actin DNA (or any other host DNA fragment) and comparison with DNA levels of the no-drug control is a relative measurement of the inhibitory effect of the test compound on cellular DNA polymerases

With the availability of both the HCV FCt data and the rDNA □Ct, a specificity parameter can be introduced. This parameter is obtained by subtracting both □Ct values from each other. This results in ΔCt values; a value above 0 means that there is more inhibitory effect on the replicon encoded polymerase, a ΔCt value below 0 means that the host rDNA levels are more affected than the replicon levels. As a general rule, ΔCt values above 2 are considered as significantly different from the no-drug treatment control, and hence, is an interested compound for further evaluation. However, compounds with a ΔCt value of less than 2, but with limited molecular cytotoxicty (rDNA ΔCT between 0 and 2) may be desired.

Compounds that result in the specific reduction of HCV replicon RNA levels, but with limited reductions in cellular RNA and/or DNA levels are candidate compounds for antiviral therapy. Candidate compounds belonging to general formula group (I) were evaluated for their specific capacity of reducing Flaviviridae RNA (including HCV), and potent compounds were detected. Most studies indicate that HCV genotypes 1a and 1b are more resistant to treatment with any interferon alpha-based therapy than non-type 1 genotypes. For this reason, some doctors may prescribe longer durations of treatment for patients infected with viral genotypes 1a or 1b. Therefore, in one embodiment, gemcitabine is administered to a patient infected with HCV1a or 1b in doses effective in reducing viral load. Therefore, in one embodiment of the invention, gemcitadine is administered to a host carrying HCV genotype 1a 1b independently of interferon alpha. In a further embodiment, gemcitabine is administered to a host carrying HCV genotype 1a or 1b in combination with interferon alpha.

### Example 2

### Antiviral Activity of Gemcitabine (dFdC)

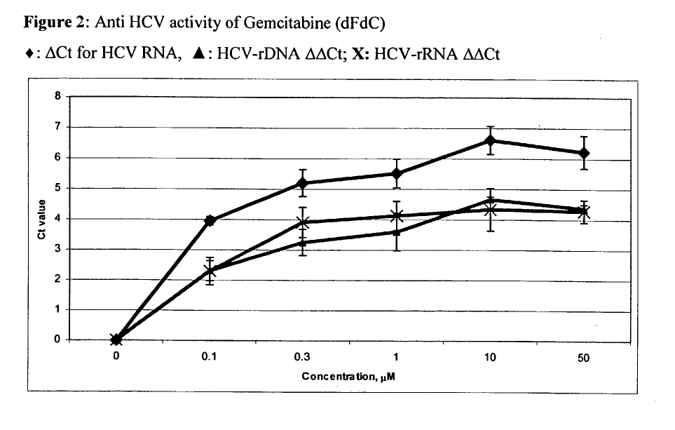

Gemcitabine was dissolved in DMSO and added to the culture media of a cellular model system of Huh7 cells harboring self-replicating HCV RNA, at final concentrations ranging from 0.1 to 50 dM. In such experiments, one way to express the antiviral effectiveness of a compound is to subtract the threshold reverse-transcriptase polymerase chain reactions (RT-PCR) cycle of the test compound with the average threshold RT-PCR cycle of the negative control. This value is called DeltaCt (ΔCt or dCt). With the availability of both the HCV ΔCt data and the rRNA ΔCt, a specificity parameter can be introduced. This parameter is obtained by subtracting both ΔCt values from each other. This results in Delta-DeltaCT values (ΔCt or ddCt). A 4-days incubation resulted in dose-dependant reduction of the replicon HCV RNA (FIG. 2). Since 3.3 Ct values equals 1-log reduction of replicon RNA, an EC₉₀ value was reached at approximately 70 nM. Further analysis of the reduction of cellular DNA levels (ribosomal DNA) or cellular RNA levels (ribosomal RNA) resulted in a dCt that expressed the inhibitory capacity of this compound on host DNA and RNA polymerases. Based on these calculations, In a cellular model system of Huh7 cells harboring self-replicating HCV RNA, gemcitabine significantly reduced HCV RNA levels (EC₅₀=0.040 μM) at a concentration below the IC₅₀ (0.240 μM). Interestingly, the inactive metabolite dFdU (7.0 μM) demonstrated similar activity to dFdC in the HCV replicon system [dCT HCV=6.39, dCt rRNA=1.96, and ddct: 4.42; (EC₅₀ and IC₅₀ data not available)].

### Example 3

### Antiviral Activity of Gemcitabine After Single Treatment in Human

A male patient exhibiting multifocal HCC, cirrhosis, and ischaemic hepatitis infected with HCV was administered 1200 mg gemcitabine HCl in 1000 minutes associated with oxaliplatine. The tolerance was acceptable, and thus the next day the patient was given a second dosage of approximately 700 mg of gemcitabine. Before the second dosage the baseline viral load was 6.49 log copies/mL. The second perfusion of gemcitabine was stopped after approximately 700 mg because of heart problems. The HCV RNA measurement eight hours after the second dosage was 4.04 log copies/mL, indicating an approximate 2.5 log drop in eight hours.

I claim:

1. A method for the treatment of a patient infected with a hepatitis C virus, comprising administering gemcitabine or its pharmaceutically acceptable salt or prodrug
(i) in an amount between 50-1300 mg/M² of host surface area
(ii) in a dosage regimen of daily for one, two, three., four, five, six or seven consecutive days followed by cessation of therapy.

2. The method of claim 1 wherein gemcitabine or its salt or prodrug is administered in an amount between 200-1000 mg/M² per day.

3. The method of claim 1 wherein gemcitabine is administered in an amount between 300-800 mg/m² per day.

4. The method of claim 1 wherein the dosage regimen is once a day for one day.

5. The method of claim 1 wherein the dosage regimen is once a day for two days.

6. The method of claim 1 wherein the dosage regimen is once a day for three days.

7. The method of claim 1 wherein the dosage regimen is once a day for four days.

8. The method of claim 1 wherein the dosage regimen is once a day for five days.

9. The method of claim 1 wherein the dosage regimen is once a day for six days.

10. The method of claim 1 wherein the dosage regimen is once a day for seven days.

11. The method of claim 1 wherein the dosage is administered intravenously.

12. The method of claim 1, wherein the therapy is ceased for at least two days.

13. The method of claim 1, wherein the therapy is ceased for at least three days.

14. The method of claim 1, wherein the therapy is ceased for at least one week.

15. The method of claim 1, wherein the therapy is ceased for at least two weeks.

16. The method of claim 1, wherein the therapy is ceased for at least three weeks.

17. The method of claim 1, wherein the therapy is ceased for at least one month.

18. A method for the treatment of a patient infected with a Flaviviridae infection, comprising administering gemcitabine or its pharmaceutically acceptable salt or prodrug (iii) in an amount between 50-1300 mg/M² of host surface area (iv) in a dosage regimen of daily for one, two, three., four, five, six or seven consecutive days followed by cessation of therapy.

19. The method of claim 18 wherein gemcitabine or its salt or prodrug is administered in an amount between 200-1000 mg/m² per day.

20. The method of claim 18 wherein gemcitabine is administered in an amount between 300-800 mg/m² per day.

21. The method of claim 18 wherein the dosage regimen is once a day for one day.

22. The method of claim 18 wherein the dosage regimen is once a day for two days.

23. The method of claim 18 wherein the dosage regimen is once a day for three days.

24. The method of claim 18 wherein the dosage regimen is once a day for four days.

25. The method of claim 18 wherein the dosage regimen is once a day for five days.

26. The method of claim 18 wherein the dosage regimen is once a day for six days.

27. The method of claim 18 wherein the dosage regimen is once a day for seven days.

28. The method of claim 18 wherein the dosage is administered intravenously.

29. The method of claim 18 wherein the therapy is ceased for at least two days.

30. The method of claim 18, wherein the therapy is ceased for at least three days.

31. The method of claim 18, wherein the therapy is ceased for at least one week.

32. The method of claim 18, wherein the therapy is ceased for at least two weeks.

33. The method of claim 18, wherein the therapy is ceased for at least three weeks.

34. The method of claim 18, wherein the therapy is ceased for at least one month.

35. A method for the treatment of Flaviviridae virus, comprising administering an antivirally effective amount of a β-D or β-L nucleoside of the structure: 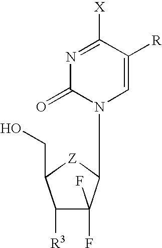
or a pharmaceutically acceptable salt or prodrug, in combination with one or more other antivirally effective agents
(i) in an amount between 50-1300 mg/m² of host surface area
(ii) in a dosage regimen of daily for one, two, three., four, five, six or seven consecutive days followed by cessation of therapy in an amount between 50-1300 mg/M²
wherein:
R is H, halogen (F, Cl, Br, I), OH, OR′, SH, SR′, NH₂, NHR′, NR₁₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C—CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR₁₂, CH═CHCO₂H, CH═CHCO₂R′;
X and is independently H, halogen, OH, OR′, OCH₃, SH, SR′, SCH₃, NH₂, NHR′, NR₁₂, CH₃;
each R′ is independently a hydrogen, lower alkyl of C₁-C₆ or lower cycloalkyl of C₁-C₆;
Z is O, S or CH₂; and
R³ is F or OH.

36. The method of claim 35 wherein X is NH₂, Z is O, R³ is OH, and R is H.

37. The method of claim 35 wherein gemcitabine or its salt or prodrug is administered in an amount between 200-1000 mg/M² per day.

38. The method of claim 35 wherein gemcitabine is administered in an amount between 300-800 mg/m2 per day.

39. The method of claim 35 wherein the dosage regimen is once a day for one day.

40. The method of claim 35 wherein the dosage regimen is once a day for two days.

41. The method of claim 35 wherein the dosage regimen is once a day for three days.

42. The method of claim 35 wherein the dosage regimen is once a day for four days.

43. The method of claim 35 wherein the dosage regimen is once a day for five days.

44. The method of claim 35 wherein the dosage regimen is once a day for six days.

45. The method of claim 35 wherein the dosage regimen is once a day for seven days.

46. The method of claim 35 wherein the dosage is administered intravenously.

47. The method of claim 35, wherein the therapy is ceased for at least two days.

48. The method of claim 35, wherein the therapy is ceased for at least three days.

49. The method of claim 35, wherein the therapy is ceased for at least one week.

50. The method of claim 35, wherein the therapy is ceased for at least two weeks.

51. The method of claim 35, wherein the therapy is ceased for at least three weeks.

52. The method of claim 35, wherein the therapy is ceased for at least one month.

53. The method of claim 35, wherein the Flaviviridae is hepatitis C virus.

54. The method of claim 18 or 35, wherein the Flaviviridae is West Nile Virus.

55. The method of claim 18 or 35, wherein the Flaviviridae is Dengue virus.

56. The method of claim 18 or 35, wherein the Flaviviridae is Bovine Viral Diarrhea Virus.

57. The method of claim 18 or 35, wherein the Flaviviridae is Border Disease Virus.

58. The method of claim 18 or 35, wherein the Flaviviridae is Yellow Fever virus.
